(19) United States
(12) Patent Application Publication
(10) Pub. No.: US 2008/0306557 A1
(43) Pub. Date: Dec. 11, 2008

(54) Inserter for a spinal implant

(76) Inventors: Moti Altarac, Irvine, CA (US); Stanley Kyle Hayes, Mission Viejo, CA (US); Tyler Jay Haskins, Oceanside, CA (US); Robert C. Gutierrez, Huntington Beach, CA (US)

Correspondence Address: RIMAS LUKAS;VERTIFLEX, INC., 1351 CALLE AVANZADO, SAN CLEMENTE, CA, 92673, US

(21) Appl. No.: 12/157,647

(22) Filed: Jun. 11, 2008

Related U.S. Application Data
(60) Provisional application No. 60/934,104, filed on Jun. 11, 2007.

(51) Int. Cl. A61F 5/00; A61B 17/00

(52) U.S. Cl. 606/86 A; 606/207; 606/206

(57) ABSTRACT

An inserter for implanting a spinal implant such as an intervertebral spacer into a spinal disc space is disclosed. The inserter comprises a jaw assembly connected to a shaft assembly that is connected to a handle assembly. The shaft assembly includes an angled portion in which the distal end of the instrument is displaced from the proximal end of the instrument affording the clinician a more unobstructed view of the operative site. The user operates the handle assembly at the proximal end to open and close the jaw assembly to thereby connect to and release from the intervertebral spacer.

### CROSS-REFERENCE TO RELATED APPLICATIONS

This application claims priority to and the benefit of U.S. Provisional Patent Application Ser. No. 60/934,104 entitled “Inserter for intervertebral spacer” filed on Jun. 11, 2007 which is incorporated herein by reference in its entirety. This application also claims priority to and is a continuation-in-part of U.S. patent application Ser. No. 12/156,857 entitled “Inserter for a spinal implant” filed on Jun. 4, 2008 which claims priority to and the benefit of U.S. Provisional Patent Application Ser. No. 60/933,538 filed on Jun. 7, 2007, all of which are hereby incorporated by reference in their entireties.

### FIELD

The present invention generally relates to medical devices, and in particular, the present invention relates to a surgical instrument for introducing spinal implants such as an intervertebral spacer into a disc space between adjacent vertebral bodies.

### BACKGROUND

Deterioration or dislocation of a spinal disc located between two adjacent vertebral bodies often results in the two adjacent vertebral bodies coming closer together. The reduced disc space height typically results in instability of the spine, decreased mobility and pain and discomfort for the patient. A common treatment is to surgically restore the proper disc space height to thereby alleviate the neurologic impact of the collapsed disc space. Typically, most surgical corrections of a disc space include at least a partial discectomy which is followed by restoration of normal disc space height and, in some instances, fusion of the adjacent vertebral bodies. Restoration of normal disc space height generally involves the implantation of a spacer and fusion typically involves inclusion of bone graft or bone graft substitute material into the intervertebral disc space to create bony fusion. Fusion rods may also be employed. Some implants further provide artificial dynamics to the spine. Such techniques for achieving interbody fusion or for providing artificial disc functions are well-known in the art.

One problem, among others, with inserting an implant, for example, is associated with patient anatomy. Inserting and positioning the implant in the space between adjacent vertebrae can be difficult or time consuming if the bony portions are spaced too close together, or if the adjacent tissue, nerves or vasculature impedes access to or placement of the implant in the space between the bony portions. Furthermore, maintenance of distraction of the space during insertion of the implant requires additional instruments in the operative space which can make the procedure more invasive and impede access and visibility during implant insertion and thereby make the procedure more difficult.

Another difficulty of implant insertion is related to the point of access to the damaged disc space which may be accomplished from several approaches to the spine with each approach having different associated difficulties. One approach is to gain access to the anterior portion of the spine through a patient's abdomen. For an anterior approach, extensive vessel retraction is often required and many vertebral levels are not readily accessible from this approach. Another approach is a posterior approach. This approach typically requires that both sides of the disc space on either side of the spinal cord be surgically exposed, which may require a substantial incision or multiple access locations, as well as extensive retraction of the spinal cord. Yet another approach is a postero-lateral approach to the disc space. The posterior-lateral approach is employed in a posterior lumbar interbody fusion (PLIF) or transforaminal lumber interbody fusion (TLIF) procedure which may be performed as an open technique which requires making a larger incision along the middle of the back. Through this incision, the surgeon then cuts away, or retracts, spinal muscles and tissue to access the vertebrae and disc space. The TLIF procedure may also be performed as a minimally invasive or as an extreme lateral interbody fusion (XLIF) procedure that involves a retroperitoneal transpoas approach to the lumbar spine as an alternative to “open” fusion surgery. In the minimally invasive procedure, the surgeon employs much smaller incisions, avoids disrupting major muscles and tissues in the back and reduces the amount of muscle and tissue that is cut or retracted. As a result, blood loss is dramatically reduced and these minimally invasive benefits also lead to shorter hospital stays and quicker patient recovery times. The aforementioned and various other difficulties associated with the point of access to the damaged disc space and the need to navigate an implant insertion instrument through the point of access further place demands on insertion instrument design. Therefore, there remains a need for improved insertion instruments, implants and techniques for use in any one or more types of approaches to the disc space that facilitate and provide for effective insertion while saving time, minimizing the degree of invasiveness for the patient and complementing surgeon skill demands.

### SUMMARY

According to one aspect of the invention, an inserter for implanting a spinal implant is disclosed. The instrument includes a shaft assembly connected to a jaw assembly at one end and to a handle assembly at the other end. The shaft assembly has an angled portion and includes an inner shaft and an outer shaft. The handle assembly is connected to the shaft assembly such that the handle assembly is operable to open and close the jaw assembly to thereby connect to and release the spinal implant.

According to another aspect of the invention, an inserter for a spinal implant is provided. The instrument includes a jaw assembly, a shaft assembly and a handle assembly. The shaft assembly is connected to the jaw assembly. The shaft assembly includes an inner shaft and a distal shaft. The inner shaft has a distal end configured to engage the jaw assembly. The inner shaft is located in the outer shaft such that the inner shaft is movable with respect to the outer shaft. The distal end of the outer shaft is connected to the jaw assembly such that the jaw assembly is movable with respect to the outer shaft. The handle assembly is connected to the shaft assembly. The handle assembly includes a first portion connected to the second portion such that the second portion is movable with respect to the first portion. The first portion is connected to the proximal end of the outer shaft and the second portion is connected to the proximal end of the inner shaft. The inner shaft is operable via the second portion to open and close the jaw assembly.

### BRIEF DESCRIPTION OF THE DRAWINGS

The invention is best understood from the following detailed description when read in conjunction with the accompanying drawings. It is emphasized that, according to common practice, the various features of the drawings are not to-scale.

On the contrary, the dimensions of the various features are arbitrarily expanded or reduced for clarity.

FIG. 1a illustrates a top view of an inserter according to the present invention.

FIG. 1b illustrates a cross-sectional view of the inserter of FIG. 1 according to the present invention.

FIG. 2a illustrates a perspective view of a jaw piece of a jaw assembly of an inserter according to the present invention.

FIG. 2b illustrates a top cross-sectional view of the jaw piece of FIG. 2a of an inserter according to the present invention.

FIG. 3a illustrates a perspective view of an outer shaft of a shaft assembly of an inserter according to the present invention.

FIG. 3b illustrates a top view of the outer shaft of FIG. 3a according to the present invention.

FIG. 3c illustrates a side view of the outer shaft of FIG. 3a according to the present invention.

FIG. 4 illustrates a top view of an inner shaft of a shaft assembly of an inserter according to the present invention.

FIG. 5 illustrates a cross-sectional view of a handle of a handle assembly of an inserter according to the present invention.

FIG. 6 illustrates a cross-sectional view of a knob of a handle assembly according to the present invention.

FIG. 7a illustrates a top and cross-sectional view of a spacer in juxtaposition with an inserter according to the present invention.

FIG. 7b illustrates a top cross-sectional view of a spacer connected to an inserter according to the present invention.

### DETAILED DESCRIPTION

Before the subject devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a spinal segment” may include a plurality of such spinal segments and reference to “the screw” includes reference to one or more screws and equivalents thereof known to those skilled in the art, and so forth.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention is described in the accompanying figures and text as understood by a person having ordinary skill in the field of spinal implants and related instrumentation.

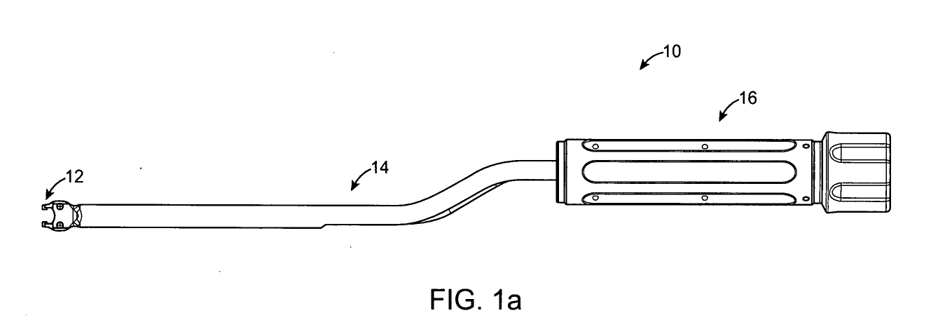

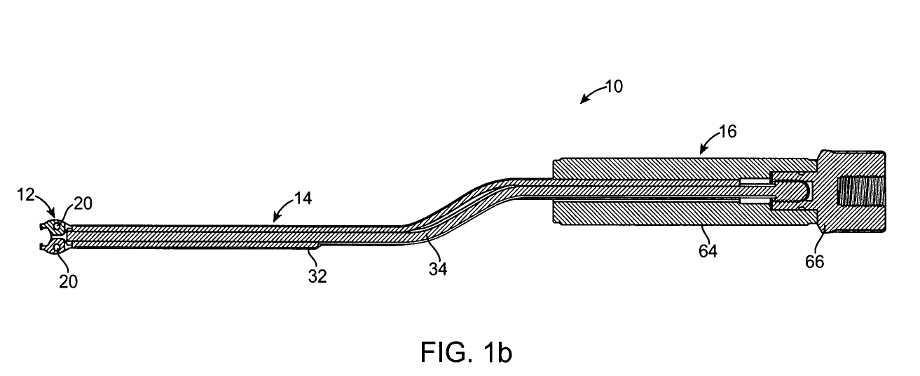

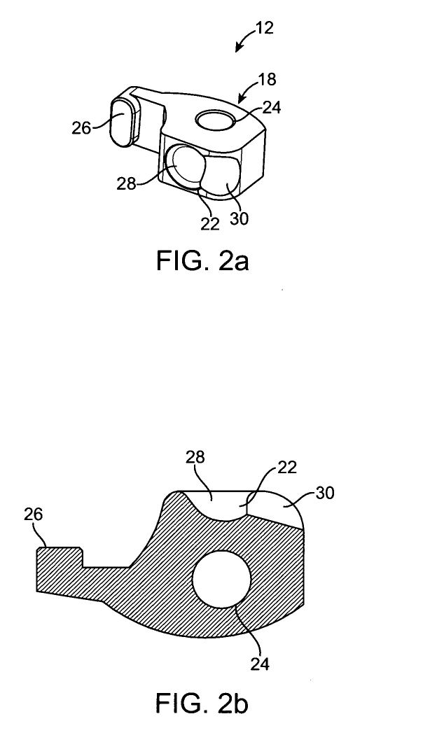

Referring now to FIGS. 1a and 1b, there are shown top and cross-sectional views, respectively, of an inserter 10 for inserting an intervertebral spacer into a disc space between two adjacent vertebral bodies. The inserter 10 includes a jaw assembly 12, a shaft assembly 14 and a handle assembly 16. The shaft assembly 14 is connected to the jaw assembly 12 and the handle assembly 16. Turning now to FIGS. 2a and 2b, there are shown perspective and cross-sectional views, respectively, of the jaw assembly 12 according to the present invention. The jaw assembly 12 includes a two jaw pieces 18 (one jaw piece is shown in FIGS. 2a and 2b), and two fasteners 20 (shown in FIG. 1b). Each jaw piece 18 includes a jaw pin receiving portion 22, a bore 24 for receiving a fastener 20 and spacer engaging features 26. The spacer engaging features 26 are extending features configured to engage the interbody spacer (not shown). In one variation, the features 26 are projections configured to be inserted into complementary shaped apertures in the interbody spacer. The jaw pin receiving portion 22 includes a first receiving portion 28 and a scalloped portion 30.

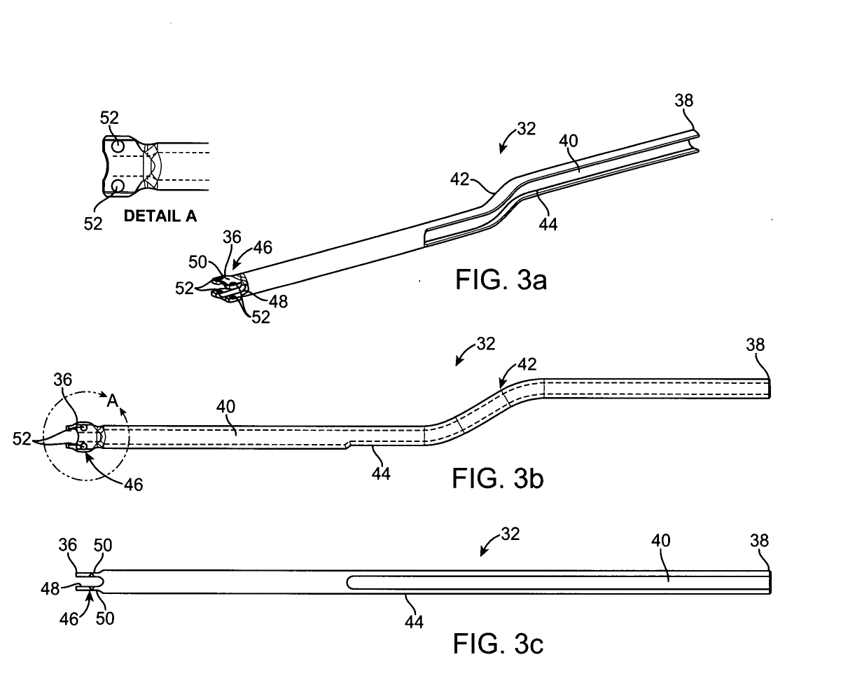

Referring back to FIG. 1b, the shaft assembly 14 includes an outer shaft 32 and an inner shaft 34. The inner shaft 34 is disposed inside the outer shaft 32. The outer shaft 32 will now be discussed in reference to FIGS. 3a, 3b and 3c.

Turning now to FIGS. 3a, 3b and 3c, there are shown perspective, top and side views, respectively, of the outer shaft 32 according to the present invention. The outer shaft 32 includes a distal end 36 and a proximal end 38. The outer shaft 32 is configured as a tube having a central bore 40 opening at and extending between the distal and proximal ends 36, 38. In one variation, the outer shaft 32 includes an angled portion 42 imparting the outer shaft 32 with a curve, bend or bayonet-like appearance. The bayonet shape permits the working distal end 36 to be displaced from the proximal handling end 38. The displacement of the working distal end 36 from the proximal handling end 38 by a distance eases installation of the implant and helps keep the working distal end 36 substantially unobstructed by the instrument's proximal end when viewed from the proximal end 38 along the longitudinal axis of the distal end 36. In another variation, the outer shaft 32 is not angled and is a substantially straight tube. In one variation, the outer shaft 32 includes an open portion 44 that opens to and extends from the proximal end 38 towards the distal end 36. The open portion 44 comprises a section of the outer shaft 32 in which the at least a portion of the wall is removed.

Still referencing FIGS. 3a, 3b and 3c, the distal end 36 of the outer shaft 32 includes a jaw assembly receiving portion 46 configured to receive and connect to the jaw assembly 12. The jaw assembly receiving portion 46 includes a slot 48 and two substantially flattened portions 50 in substantial parallel orientation with respect to one another. Each flattened portion 50 includes two aligned bores 52 for receiving fasteners 20.

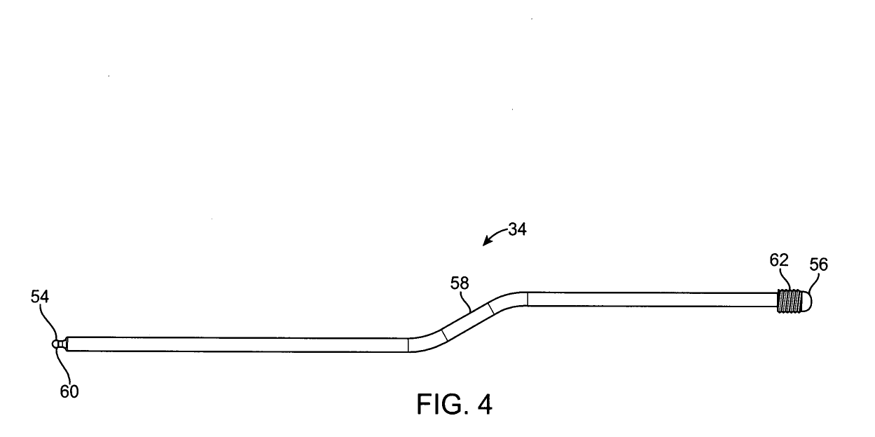

Turning now to FIG. 4, there is shown an inner shaft 34 according to the present invention. The inner shaft 34 includes a distal end 54 and a proximal end 56.

The inner shaft 34 is configured to be substantially cylindrical in shape. In one variation, the inner shaft 34 includes an angled portion 58 imparting the inner shaft 34 with a curve, bend or bayonet-like appearance. The angled portion 58 of the inner shaft 34 is configured to conform to the shape of an angled outer shaft 32 such that the angled inner shaft 34 fits inside an angled outer shaft 32. In another variation, the inner shaft 34 is not angled and is substantially straight and configured to fit within an outer shaft 32 that is also substantially straight. The inner shaft 34 includes a pin 60 at the distal end 54 configured to engage the jaw assembly 12 and to be received in the pin receiving portions 22 of each jaw piece 18. The pin 60 has a bulbous head or spherically-shaped head connected to a neck portion as shown in FIG. 4. Other suitable and functional shapes for the pin 60 are within the scope of the present invention and include any polyhedron or partial polyhedron, cube or partial cube, sphere or partial sphere, cylinder or partial cylinder, a prism or partial prism, cylinder or partial cylinder, and any shape with an angled surface or any shape having a surface angled with respect to the inner shaft. The proximal end 56 of the inner shaft 34 includes a threaded portion 62 configured for threaded engagement with the handle assembly 16. The shaft assembly 14 is assembled by inserted the inner shaft 34 into the outer shaft 32.

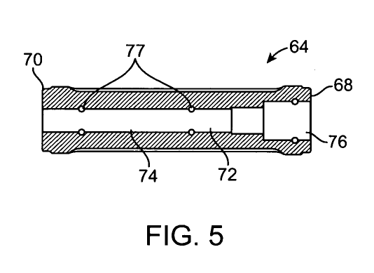

Turning briefly back to FIG. 1b, the handle assembly 16 includes a handle 64 and a knob 66. The handle 64 will now be discussed in reference to FIG. 5.

Referring now to FIG. 5, there is shown a cross-sectional view of the handle 64 of the handle assembly 16. The handle 64 includes a proximal end 68 and a distal end 70. The handle 64 has an outer gripping surface and is substantially cylindrical in shape. The handle 64 includes a shaft assembly receiving portion 74 at the distal end 70 configured to connect with at least a portion of the shaft assembly 14. The handle also includes a knob receiving portion 76 at the proximal end 68 configured to connect to the knob 66. In one variation, the shaft assembly receiving portion 74 and the knob receiving portion 76 form a central bore 72 of varied diameter opening to and extending between the proximal and distal ends 68, 70 as shown in FIG. 5. The handle 64 also includes at least one pin slot 77 for the insertion of pins (not shown) to securely connect the handle 64 to the outer shaft 32.

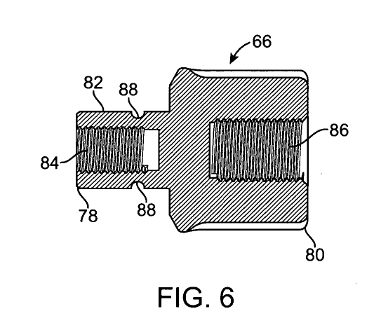

Referring now to FIG. 6, there is shown a cross-sectional view of the knob 66 of the handle assembly 16. The knob 66 has a distal end 78 and a proximal end 80.

The distal end 78 includes an engaging portion 82 configured to connect with the handle 64 and with the inner shaft 34. The engaging portion 82 includes a male member having an interior threaded bore 84 for connecting with the threaded portion 62 of the inner shaft 34. The interior threaded bore 84 opens at the distal end 84 and extends inwardly towards the proximal end 80. The outer surface of the male member engaging portion 82 is sized to be received in the knob receiving portion 76 of the handle 64 and includes recesses 88 for receiving locking pins for connecting the knob 66 to the handle 64. The proximal end 80 of the knob 66 has a larger cross-section and includes an interior threaded bore 86 opening at the proximal end 80 and extending inwardly towards the distal end 78. The threaded bore 86 serves as a connection point for a slap hammer attachment (not shown) permitting use of a slap hammer to aid in removing the inserter 10 from tight intervertebral spaces.

The assembly of the inserter 10 will now be discussed. The inner shaft 34 is inserted into the outer shaft 32. Pins 60 of the inner shaft 34 are located in the pin receiving portions 22 of each jaw piece 18. Fasteners 20 are inserted into the aligned bores 52 of the outer shaft and bores 24 of the jaw pieces 18 and swaged thereto to secure the jaw pieces 18 to the outer shaft 32 capturing the pin 60 of the inner shaft 34 in between the jaw pieces 18 such that the jaw pieces 18 are capable of movement with respect to the outer shaft 32 and about fasteners 20. At the proximal end, the threaded portion 62 of the inner shaft 34 is threadingly engaged inside bore 84. The outer and inner shafts 32, 34 are passed into the central bore 72 of the handle 64. Pins are passed into apertures 77 of the handle 64 to secure the handle 64 and outer shaft 32 together such that the inner shaft 34 is permitted to move with respect to the outer shaft 32. Other pins are passed into apertures 77 to engage recesses 88 to prevent the knob 66 from falling out yet permitting it to rotate with respect to the handle 64.

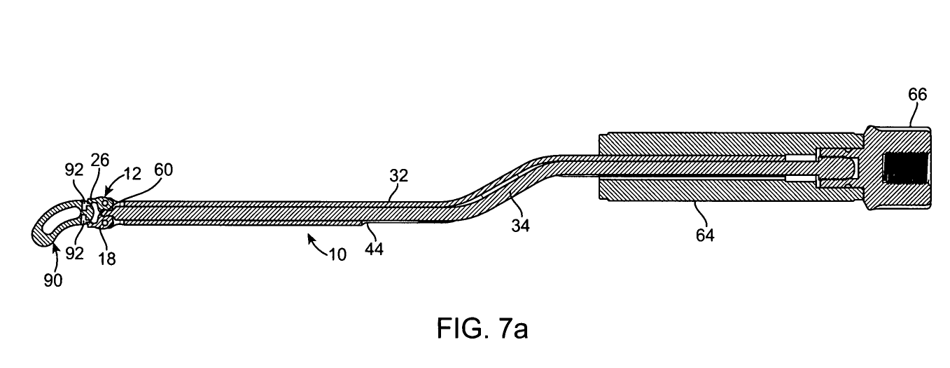

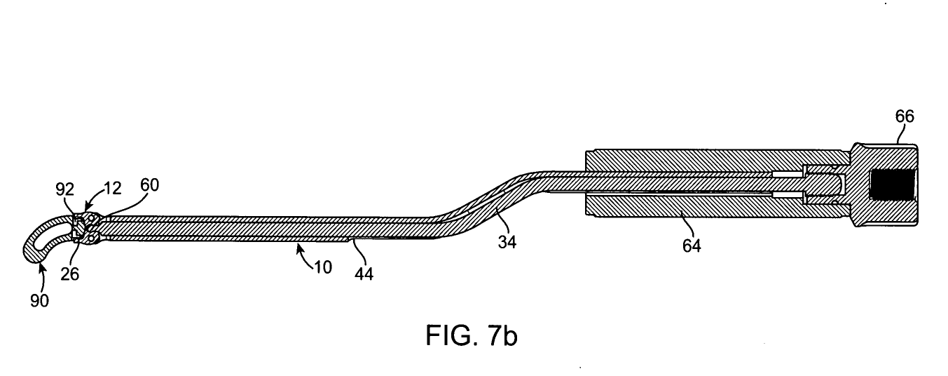

Operation of the inserter instrument 10 will now be discussed with initial reference to FIGS. 7a and 7b. Referring firstly to FIG. 7a, an interbody spacer 90 having engaging apertures 92 is shown in juxtaposition with the inserter 10 with the jaw assembly 12 in an open position in which the jaw pieces 18 are spread apart, pivoted about their respective connecting fasteners 20. The typical spacer 90 includes a body formed by a wall extending about a central cavity. The cavity extends between and opens at an upper bearing surface and a lower bearing surface. The upper and lower bearing surfaces contact the adjacent vertebral endplates to support the adjacent vertebrae when the spacer is implanted into the spinal disc space. The bearing surfaces include grooves formed to facilitate engagement with the vertebral endplates and resist the spacer from migrating within the disc space. The spacer includes a convexly curved anterior wall and an opposite concavely curved posterior wall. These wall portions are connected by a convexly curved leading end wall and a convexly curved trailing end. The overall shape provides a banana or kidney type shape for the spacer.

The spacer 90 includes spacer engaging apertures 92 that are shown in FIG. 7a to be aligned with the spacer engaging features 26 of the jaw assembly 12. The handle knob 66 is rotated such that the threaded engagement with the inner shaft 34 draws the inner shaft 34 proximally with respect to the handle 64 and outer shaft 32 moving the integral jaw pin 60 along with it, thereby angulating the jaws 18 about fasteners 20 into a closed position as shown in FIG. 7b. In the closed position, the spacer engaging features 26 are clamped to the spacer 90 as shown in FIG. 7b. Turning the knob 66 in reverse releases the spacer 90. Hence, the spacer 90 is released and recaptured as desired. The open portion 44 of the outer shaft 32 allows the inner shaft 34 to pass therethrough as it moves proximally and distally with respect to the outer shaft 32 as the knob 66 is turned.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

We claim:

1. An inserter for implanting a spinal implant, comprising:
a jaw assembly;
a shaft assembly connected to the jaw assembly; the shaft assembly having an angled portion and comprising an inner shaft and an outer shaft; and
a handle assembly connected to the shaft assembly such that the handle assembly is operable to open and close the jaw assembly to thereby connect to and release the spinal implant.

2. The inserter of claim 1 wherein the outer shaft includes an open portion that allows the inner shaft to pass therethrough as the handle assembly is operated to open and close the jaw assembly.

3. The inserter of claim 1 wherein the inner shaft is movable with respect to the outer shaft.

4. The inserter of claim 3 wherein the jaw assembly is movably connected to the outer shaft.

5. The inserter of claim 4 wherein movement of the inner shaft with respect to the outer shaft opens or closes the jaw assembly.

6. The inserter of claim 5 wherein the jaw assembly includes two jaw pieces movably connected to the outer shaft.

7. The inserter of claim 6 wherein the inner shaft includes a distal end configured to engage the two jaw pieces and movement of the inner shaft with respect to the outer shaft opens or closes the jaw assembly.

8. The inserter of claim 1 wherein the handle assembly includes a first portion connected to a second portion.

9. The inserter of claim 8 wherein the inner shaft is movable with respect to the outer shaft; the outer shaft is connected to the first portion and the inner shaft is connected to the second portion such that movement of the second portion moves the inner shaft with respect to the first portion and outer shaft.

10. The inserter of claim 9 wherein the jaw assembly includes two jaw pieces movably connected to the outer shaft and configured to removably attach to the spinal implant; the inner shaft includes a distal end configured to engage the two jaw pieces such that the movement of the inner shaft with respect to the outer shaft opens or closes the jaw assembly.

11. The inserter of claim 1 wherein the jaw assembly includes two movable jaw pieces configured to connect to the spinal implant and the shaft assembly includes a shaft having a distal end configured to engage the two movable jaw pieces; the shaft being movable with respect to the two jaw pieces such that relative motion of the shaft with respect to the jaw pieces moves the jaw pieces apart or together to open or close the jaw assembly for attaching or releasing the spinal implant.

12. The inserter of claim 1 wherein the inserter is configured such that the distal working end of the inserter is displaced from the proximal handle end by a distance such that the distal working end of the inserter is substantially unobstructed by the inserter's proximal end when viewed from the proximal end along the longitudinal axis of the distal end.

13. An inserter for a spinal implant comprising:
a jaw assembly;
a shaft assembly connected to the jaw assembly; the shaft assembly including
an inner shaft having a distal end configured to engage the jaw assembly and
an outer shaft; the inner shaft located in the outer shaft such that the inner shaft is movable with respect to the outer shaft; the distal end of the outer shaft being connected to the jaw assembly such that the jaw assembly is movable with respect to the outer shaft; and
a handle assembly connected to the shaft assembly; the handle assembly including a first portion connected to a second portion such that the second portion is movable with respect to the first portion; the first portion is connected to the proximal end of the outer shaft and the second portion is connected to the proximal end of the inner shaft;
wherein the inner shaft is operable via the second portion to open and close the jaw assembly.

14. The inserter of claim 13 wherein the jaw assembly includes two jaw pieces configured to attach to the spinal implant.

15. The inserter of claim 13 wherein the shaft assembly includes an angled portion.

16. The inserter of claim 13 wherein the inserter is configured such that the distal working end of the inserter is displaced from the proximal handle end by a distance such that the distal working end of the inserter is substantially unobstructed by the inserter's proximal end.

17. The inserter of claim 13 wherein the proximal end of the inner shaft is threadingly connected to the second portion such that rotation in one direction of the second portion moves the inner shaft distally and rotation in an opposite direction of the second portion moves the inner shaft proximally to open and close the jaw assembly.
